(19) United States
(12) Patent Application Publication
(10) Pub. No.: US 2002/0095034 A1
(43) Pub. Date: Jul. 18, 2002

(54) Imipenem production process

(75) Inventors: Maurizio Zenoni, Paullo (IT); Manuel Camozzi, Cene (IT); Simone Maschio, Milano (IT)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: ACS DOBFAR S.p.A., Tribiano (IT)

(21) Appl. No.: 10/032,034

(22) Filed: Dec. 31, 2001

(30) Foreign Application Priority Data
Jan. 17, 2001 (IT) MI 2001A000077

(51) Int. Cl. C07D 487/04

(52) U.S. Cl. 540/350

(57) ABSTRACT

An imipenem production process by which a compound of formula

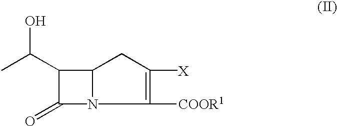

is reacted firstly with cysteamine and then with benzylformimidate to give an intermediate which is transformed into imipenem by catalytic hydrogenation.

The present invention relates to a process for producing imipenem of formula

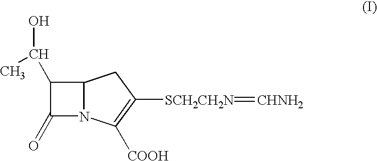

Imipenem is an antibiotic known in the literature and is described, for example, in U.S. Pat. Nos. 4,292,436, 4,374,772 and 4,894,450.

According to the most recent of these patents (U.S. Pat. No. 4,894,450) a compound of formula

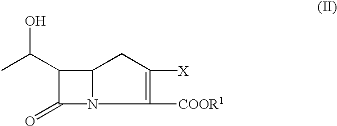

in which R¹ is p-nitrobenzyl and X is 2,4-dichlorodiphenylphosphate, is reacted in anhydrous solvent with cysteamine hydrochloride in the presence of a tertiary amine to give a solution containing a first intermediate of formula (II) in which R¹ is p-nitrobenzyl and X is —S—CH₂—CH₂—NH₂, this first intermediate is then reacted with benzylformimidate hydrochloride in the presence of said tertiary amine to give a second intermediate of formula (II) where R¹ is again p-nitrobenzyl and X is —S—CH₂—CH₂—NH—CH═NH, this second intermediate then being transformed into imipenem (I) by catalytic hydrogenation. According to U.S. Pat. No. 4,894,450 the quantities of tertiary amine used in the reaction are always in large stoichiometric excess over the quantity of compound (II), hence achieving a basic environment which lasts for all those process passages preceding the catalytic hydrogenation. In this manner both the first intermediate and the second intermediate were obtained in the form of free bases which are each soluble in the reaction environment. The imipenem (I) was then obtained by catalytic hydrogenation directly from these solutions (after simply diluting them), which contain all the by-products of the preceding reactions, with consequent difficulties in purifying the desired imipenem (I).

It has been surprisingly found that the aforesaid reactions take place very effectively (with high transformation of compound II into imipenem (I) if the base quantity is controlled such that the reaction environment is at slightly acid pH: the main result obtained is that both the first intermediate and the second intermediate precipitate in pure form, such that pure imipenem can be obtained from a solution of the second pure intermediate by catalytic hydrogenation.

The invention hence relates to a process for producing imipenem of formula

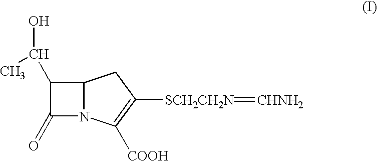

according to which a starting compound of formula

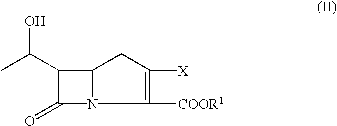

in which R¹ is a protective group for the carboxyl of a betalactam compound and X is chosen from the group consisting of diphenylphosphate, 2,4-dichlorodiphenylphosphate, diethylthiophosphate, tosylate, mesylate, is reacted in anhydrous solvent with cysteamine hydrochloride in the presence of a base to give a solution containing a first intermediate of formula (II) where X is —S—CH₂—CH₂—NH₂, which is then transformed into imipenem (I), characterised in that the quantity of said base used to obtain said first intermediate is equal to one equivalent of said starting compound (II), said anhydrous solvent being chosen from the group consisting of substituted amides, haloalkans, nitriles and ethers, from which said first intermediate precipitates with at least 97% purity in the form of hydrochloride as such, or in the form of its solvate if said substituted amides are used, said hydrochloride being isolated as first intermediate by filtration and then reacted with a compound chosen from the group consisting of C₁-C₆ alkylformimidate hydrochlorides and C₇-C₁₂ arylalkylformimidate hydrochlorides to give a protected compound which is subjected to deprotection treatment, whatever the sequence of these two latter operations, to give imipenem (I).

Advantageously, before being subjected to said deprotection treatment said protected compound is treated with a hydrophobic resin.

Preferably said base is a tertiary amine; R¹ is p-nitrobenzyl; the anhydrous solvent is chosen from the group consisting of N-methylpyrrolidinone, N-ethylpyrrolidinone, N,N-dimethylacetamide, dichloromethane, acetonitrile and tetrahydrofuran; the formimidate hydrochloride is benzylformimidate hydrochloride; and X is diphenylphosphate.

As stated above, the two latter operations leading to the obtaining of the imipenem (I) can be carried out in either order.

According to one of the two methods said hydrochloride of the first intermediate is firstly dissolved in the presence of one base equivalent in a solvent chosen from the group consisting of acetonitrile, dichloromethane and tetrahydrofuran, then reacted with the compound chosen from the group consisting of C₁-C₆ alkylformimidate hydrochlorides and C₇-C₁₂ arylalkylformimidate hydrochlorides to give a second intermediate of formula (II) in which R¹ is p-nitrobenzylester and X is —S—CH₂—CH₂—NH—CH═NH which precipitates in the form of hydrochloride with a purity of at least 95% and is transformed into imipenem (I) by deprotection by means of catalytic hydrogenation in accordance with the known art.

According to the other method, which is alternative but equivalent to the aforestated, said first intermediate is firstly deprotected by catalytic hydrogenation in the presence of a buffer chosen from the group consisting of phosphates, morpholinoalkylsulphonic acids and N-alkyl morpholines, and then transformed into imipenem (I) by reaction with a compound chosen from the group consisting of C₁-C₆ alkylformimidate hydrochlorides and C₇-C₁₂ arylalkylformimidate hydrochlorides, in accordance with the known art.

The invention also relates to the product of formula (I), where R¹ is p-nitrobenzyl and X is —S—CH₂—CH₂—NH₂ in the form of hydrochloride, or hydrochloride solvated with a solvating agent chosen from the group consisting of N-methylpyrrolidinone, N-ethylpyrrolidinone and N,N-dimethylacetamide; preferably the solvating agent is present in a quantity between 5% and 50% by weight; the product itself has a purity of at least 97%. Finally it should be noted that also the aforesaid second intermediate is a new compound in the form of a solid having a purity exceeding 95%.

If the anhydrous solvent used together with the cysteamine hydrochloride is dichloromethane, acetonitrile or tetrahydrofuran, the first intermediate is obtained in the form of hydrochloride, whereas if the anhydrous solvent is a substituted amide the first intermediate is obtained as hydrochloride solvate.

It can also be noted that the procedure for transforming the deprotected first intermediate into imipenem (I) can be carried out in accordance with the known art described in U.S. Pat. No. 4,374,772.

The following examples provide non-limiting illustrations of the present invention,

### EXAMPLE 1

### Thienamycin p-nitrobenzylester hydrochloride (N-methylpyrrolidinone solvate)

16.3 ml of ethyldiisopropylamine and 16.5 ml of diphenylphosphorohydrochloride are dropped into a solution of (3R,5R,6S)-2-oxo-6-[(1 R)-1-hydroxyethyl]carbapenem-3-carboxylic acid p-nitrobenzylester (25 g, 71.8 mmoles) in 125 ml of N-methylpyrrolidinone at 0° C. in an N₂ atmosphere. After 1.5 hours at 0° C. a solution of cysteamine hydrochloride (9.25 g) in methylpyrrolidinone is dropped in, followed by 9.0 ml of ethyldiisopropylamine.

After 1 hour thienamycin p-nitrobenzylester hydrochloride begins to precipitate, the mixture is left under agitation for 1 hour at 0° C., then acetonitrile is added leaving the mixture under slow agitation for 1 hour. The precipitate is filtered off under a stream of N₂ and washed with acetonitrile to obtain, after drying under vacuum, 28 g of a first intermediate of formula (II) consisting of thienamycin p-nitrobenzylester hydrochloride in the form of N-methylpyrrolidinone solvate (white solid). Yield: 74%.

H¹-NMR (300 MHz) (DMSO-d₆)-δ ppm: 1.15(3H, d, J=5.88), 3.07(4H, m), 3.3(3H, m), 3.97(1H, m), 4.18(1H, m), 5.16(1H, d, J=5, 16), 5.37(2H, Ab_{q}, J=13.98), 8.14(2H, bs), 7.72-8.25(4H, AA′XX′).

N-methylpyrrolidinone: 1.9(1.65H, m), 2.17(1.65H, t), 2.69(2.44H, s), 3.33(1.65H, m).

### EXAMPLE 2

### Thienamycin p-nitrobenzylester hydrochloride (N,N-dimethylacetamide solvate)

5.28 ml of ethyidiisopropylamine and 5.27 ml of diphenylphosphorohydrochloride are dropped into a solution of (3R,5R,6S)-2-oxo-6-[(1 R)-1-hydroxyethyl]carbapenem-3-carboxylic acid p-nitrobenzylester (8.77 g, 25.2 mmoles) in 100 ml of acetonitrile at 0° C. in an N₂ atmosphere.

After 3 hours at 0° C. a solution of cysteamine hydrochloride (3.25 g, 25.2 mmoles) in DMAC is dropped in, followed by 3.24 ml of ethyldiisopropylamine.

The mixture is diluted with 100 ml of acetonitrile and left under slow agitation for 3 hours. The precipitate is filtered off under a stream of N₂ and washed with acetonitrile to obtain, after drying under vacuum, 6.6 g of a first intermediate of formula (II) consisting of thienamycin p-nitrobenzylester hydrochloride in the form of solvate (white solid). Yield: 52%.

H¹-NMR (300 MHz) (DMSO-d₆)-δ ppm: to the thienamycin p-nitrobenzylester hydrochloride signals listed in Example 1 are added the following signals typical of N,N-dimethylacetamide: 1.95(2H, s), 2.78(2H, s), 2.94(2H, s).

### EXAMPLE 3

### Thienamycin p-nitrobenzylester hydrochloride (N-ethylpyrrolidinone solvate)

Operating as in Example 1, but using N-ethylpyrrolidinone instead of N-methylpyrrolidone the corresponding first intermediate is obtained, with a yield of 52%.

H¹-NMR (300 MHz) (DMSO-d₆)-δ ppm: to the thienamycin p-nitrobenzylester hydrochloride signals listed in Example 1 are added the following signals typical of N-ethylpyrrolidinone: 0.97(3H, t, J=7.35), 1.9(2H, m), 2.2(2H, t), 3.18(2H, q), 3.33(2H, m).

### EXAMPLE 4

### Displacement of thienamycin p-nitrobenzylester hydrochloride solvate

2.0 g of thienamycin-PNB hydrochloride solvate (obtained as in Example 1) are suspended in 50 ml of CH₂Cl₂ and left under agitation for 3 hours at T=20° C.

The precipitate is filtered off under a stream of N₂ and washed with 10 ml of CH₂Cl₂. 1.57 g of thienamycin p-nitrobenzylester hydrochloride are obtained. Yield=96.2%

HPLC titer=91% as base.

### EXAMPLE 5

### Imipenem p-nitrobenzylester hydrochloride

0.4 ml of ethyidiisopropylamine are dropped into a suspension of thienamycin p-nitrobenzylester hydrochloride (1.0 g, 1.7 mmoles) in 100 ml of acetonitrile at 0° C. under a stream of N₂, leaving the mixture under agitation until completely dissolved.

Benzylformimidate hydrochloride (790 mg) is added, and after 1.5 hours the mixture is diluted with 50 ml of diisopropylether and left under slow agitation at 0° C. for 1 hour.

The precipitate is filtered off under a stream of N₂ and washed with acetonitrile, then dried to obtain 700 mg of the second intermediate of formula (II), consisting of imipenem p-nitrobenzylester hydrochloride. Yield: 80%.

H¹-NMR (DMSO-d₆)-δ ppm: 1.14(3H, d, J=6.6), 3.11(2H, m), 3.58(2H, m), 3.2-3.5(3H, m), 3.96(1H, m), 4.19(1H, m), 5.18(0.94H, bs) 5.37(2H, Ab_{q}), 7.7(2H, arom., J=8.82), 8.24(2H, arom., d, J=8.82), 7.86(1H, s), 9.41(2H, bs).

The second intermediate obtained in this manner is dissolved in a mixture of water and organic solvent and transformed into imipenem (I) by catalytic hydrogenation by known methods, for example as described in Example 3 of U.S. Pat. No. 4,894,450.

### EXAMPLE 6

### Imipenem p-nitrobenzylester hydrochloride

22.2 ml (129 mmoles) of DIEA are dropped into a suspension consisting of 55 g (titer 75%, 101.3 mmoles) of thienamycin pNB hydrochloride in 830 ml of methylene chloride, maintaining the temperature at 0° C. 275 ml of methanol are then added at the same temperature.

On termination of the addition the mixture is left under agitation for 15 min. to obtain a solution which is cooled to −10° C. 22 g (128 mmoles) of benzylformimidate hydrochloride are added to this solution while maintaining the temperature at −10° C. On termination of the addition the solution is heated to +10° C. and left under agitation for 30 min. HPLC monitoring shows the thienamycin pNB hydrochloride consumption and the formation of imipenem pNB hydrochloride. 500 ml of deionized water are added to the organic solution at the end of synthesis to obtain phase separation.

The aqueous phase containing the imipenem pNB hydrochloride intermediate shows an HPLC-monitored yield of 87%. This is used either as such or after treatment with hydrophobic resin, in accordance with already known processes to give the imipenem monohydrate crystalline product with a yield of 20%, calculated on the thienamycin pNB hydrochloride.

### EXAMPLE 7

### Imipenem pNB hydrochloride

9.4 ml (55 mmoles) of DIEA are dropped into a solution consisting of 10 g (28.7 mmoles) of (3R,5R,6S)-2-oxo-6-[(1 R)-1-hydroxyethyl]-carbapenem in 100 ml of methylene chloride, maintaining the temperature at 0° C. After the addition the mixture is left under agitation until a solution is obtained, while maintaining the temperature at 0° C.

6.5 ml (31.35 mmoles) of diphenyl chloro phosphate are then added at the same temperature. The solution is left under agitation at 0° C. for one hour.

6.4 g (56,3 mmoles) of cysteamine hydrochloride are added in portions to the solution thus obtained at a temperature of 0° C., followed by 4.7 ml (27.4 mmoles) of DIEA.

The suspension obtained is maintained under agitation at 0/5° C. for 4 hours.

HPLC monitoring shows the formation of thienamycin pNB hydrochloride. 30 ml of methanol are added to the methylene solution, maintaining the temperature at 0° C.

After this addition the mixture is maintained under agitation for one hour to obtain a solution which is then cooled to −10° C.

4.7 ml (27.45 mmoles) of DIEA and 4.6 g (26.8 mmoles) of benzyl formimidate hydrochloride are then added to this solution in that order. The suspension obtained is heated to 15° C. and left under agitation for a time of 30 minutes.

HPLC monitoring shows the formation of imipenem pNB hydrochloride (yield 75% calculated on the starting acid).

The methylene phase was treated in accordance with already known processes to give the imipenem monohydrate product (yield 20% calculated on the starting acid).

### EXAMPLE 8

### Imipenem hydrochloride

6.8 g of monoprotected thienamycin hydrochloride are dissolved in 150 ml of 0.2 M aqueous KH₂PO₄, 100 ml of THF and 3 ml of isopropyl alcohol, 1.4 g of 10% Pd/C are added and hydrogenation is carried out at room temperature and 80 psi for 1 hour.

The catalyst is filtered off and the mixture washed with 3×50 ml of ethyl acetate.

The aqueous phase is reacted with alkyl or arylalkylformimidates to give imipenem in accordance with known methods, as for example in U.S. Pat. 4,374,772.

What we claim is:

1. A process for producing imipenem of formula 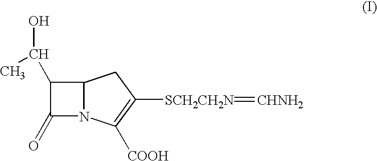
according to which a starting compound of formula 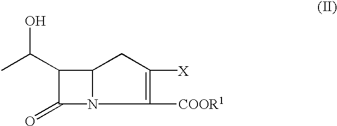
in which R¹ is a protective group for the carboxyl of a betalactam compound and X is chosen from the group consisting of diphenylphosphate, 2,4-dichlorodiphenylphosphate, diethylthiophosphate, tosylate, mesylate, is reacted in anhydrous solvent with cysteamine hydrochloride in the presence of a base to give a solution containing a first intermediate of formula (II) where X is —S—CH₂—CH₂—NH₂, which is then transformed into imipenem (I), wherein the quantity of said base used to obtain said first intermediate is equal to one equivalent of said starting compound (II), said anhydrous solvent being chosen from the group consisting of substituted amides, haloalkans, nitriles and ethers, from which said first intermediate precipitates with at least 97% purity in the form of hydrochloride as such, or in the form of its solvate if said substituted amides are used, said hydrochloride being isolated as first intermediate by filtration and then reacted with a compound chosen from the group consisting of C₁-C₆ alkylformimidate hydrochlorides and C₇-C₁₂ arylalkylformimidate hydrochlorides to give a protected compound which is subjected to deprotection treatment, whatever the sequence of these two latter operations, to give imipenem (I).

2. A process as claimed in claim 1, wherein before being subjected to said deprotection treatment said protected compound is treated with a hydrophobic resin.

3. A process as claimed in claim 1, wherein said base is a tertiary amine.

4. A process as claimed in claim 1, wherein R¹ is p-nitrobenzyl.

5. A process as claimed in claim 1, wherein said anhydrous solvent is chosen from the group consisting of N-methylpyrrolidinone, N-ethylpyrrolidinone, N,N-dimethylacetamide, dichloromethane, acetonitrile and tetrahydrofuran

6. A process as claimed in claim 1, wherein formimidate hydrochloride is benzylformimidate hydrochloride.

7. A process as claimed in claim 1, wherein X is diphenylphosphate.

8. A process as claimed in claim 1, wherein hydrochloride of the first intermediate is firstly dissolved in the presence of a base equivalent in a solvent chosen from the group consisting of acetonitrile, dichloromethane and tetrahydrofuran, then reacted with the compound chosen from the group consisting of C₁-C₆ alkylformimidate hydrochlorides and C₇-C₁₂ arylalkylformimidate hydrochlorides to give a second intermediate of formula (II) in which R¹ is p-nitrobenzylester and X is —S—CH₂—CH₂—NH—CH═NH which precipitates in the form of hydrochloride with a purity of at least 95% and is transformed into imipenem (I) by deprotection by means of catalytic hydrogenation in accordance with the known art.

9. A process as claimed in claim 1, wherein said first intermediate is firstly deprotected by catalytic hydrogenation in the presence of a buffer chosen from the group consisting of phosphates, morpholinoalkylsulphonic acids and N-alkyl morpholines, and then transformed into imipenem (I) by reaction with a compound chosen from the group consisting of C₁-C₆ alkylformimidate hydrochlorides and C₇-C₁₂ arylalkylformimidate hydrochlorides, in accordance with the known art.

10. A product of formula (II) where R¹ is p-nitrobenzyl and X is —S—CH₂—CH₂—NH₂ in the form of hydrochloride, or hydrochloride solvated with a solvating agent chosen from the group consisting of N-methylpyrrolidinone, N-ethylpyrrolidinone and N,N-dimethylacetamide.

11. A product as claimed in claim 10, wherein said solvating agent is present in a quantity between 5% and 50% by weight.

12. A product as claimed in claim 10, wherein having a purity of at least 97%.

13. The second intermediate obtained by the process of claim 8, wherein consisting of a solid having a purity exceeding 95%.
